(19) United States
(12) Patent Application Publication
(10) Pub. No.: US 2005/0102708 A1
(43) Pub. Date: May 12, 2005

(54) Animal model simulating neurologic disease

(76) Inventors: Laurent Lecanu, McLean, VA (US); Vassilios Papadopoulos, North Potomac, MD (US); Janet Greeson, Las Vegas, NV (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/798,081

(22) Filed: Mar. 11, 2004

Related U.S. Application Data
(60) Provisional application No. 60/453,886, filed on Mar. 12, 2003.

(51) Int. Cl. A01K 67/027

(52) U.S. Cl. 800/8; 800/18

(57) ABSTRACT

The present invention relates to the development of a pharmacological non-human animal model that associates memory loss to histopathological features found in the brain of a subject having Alzheimer's Disease. In one embodiment, a four-week continuous infusion of a Fe²⁺, Aβ₄₂ and buthionine sulfoximine (FAB) solution in the left ventricle of young adult Long-Evans rats induced memory impairment accompanied by increased hyperphosphorylated Tau protein levels in cerebrospinal fluid. Brains from treated animals displayed neuritic plaques, tangles, neuronal loss, astrogliosis and microgliosis in hippocampus and cortex. The present invention may be utilized in evaluating preventive, therapeutic and diagnostic means for neurologic diseases.

### RELATED APPLICATIONS DATA

This application claims priority to U.S. Provisional Application No. 60/453,886, filed Mar. 12, 2003.

### BACKGROUND OF INVENTION

Alzheimer's disease (“AD”) is the most common dementia occurring in elderly, affecting about 10% of the population over 65 years old and about 40% of the population over 80 years old. AD associates memory impairment to neurohistological modifications, the two hallmarks of the disease being the formation of neuritic plaques due to β₄₂-amyloid peptide (Aβ₄₂) aggregation and neurofibrillary tangles (“NFT”) secondary to the Tau protein hyperphosphorylation. During the last decade, several hypotheses emerged to explain the physiopathology of AD. The main current hypotheses on the origin of the disease are: amyloidogenesis (Hardy J D and Higgins G A (1992) Science, 256(5054): 185-185); disruption of calcium homeostasis (Kachaturian Z S (1987) Neurobiol Aging, 8(4): 345-346); energetic failure (Beal M F (1992) Ann Neurol, 31(2): 119-30); induction of oxidative stress (Volicer L and Crino P B (1990) Neurobiol Aging, 11(5): 567-571); and more recently, the hyperphosphorylation of the Tau protein (Mudher A and Lovestone S (2002) TiNS, 25(1): 22-26). Despite the large amount of data generated during the testing of these hypotheses, the mechanisms underlying the origin and the events responsible for the progression of AD remain elusive. For example, various drugs developed based on the above mentioned hypotheses have not demonstrated any significant effect in clinical trials.

The lack of a relevant animal model simulating AD pathophysiology is also at the source of the current weakness in developing successful therapies for AD. The development of such a model is a pre-requisite to the development of any preventive, therapeutic and diagnostic means. In an effort to analyze and identify the different factors triggering and contributing to AD, different transgenic models carrying Amyloid Protein Precursor (APP) mutations have been constructed in mice (Janus C, et al. (2000) Biochim Biophys Acta, 1502(1): 63-75; Janus C and Westaway D (2001) Physiol Behav, 73(5): 873-886). Although these models provided useful data regarding the disease, they do not mirror the pathophysiology of human AD. These models are based on genetic modifications of the APP gene that exists in human. APP mutations represent only a minor portion (less than 5%) of the total number of AD patients. In other words, these transgenic models, although they may reflect the pathophysiology of the familial AD, a genetic disease, they do not represent the common sporadic AD that represents approximately 95% of AD cases. Moreover, because of the genetically modified background of these transgenic animals the reliability of the behavior data must still be questioned as nothing is known about the neurological developmental consequences and compensatory effect of the integration of a transgene/promoter construct in a mouse. In addition, the lack of NFT and of neuronal losses further distinguishes this animal model from human AD.

Various attempts have also been made in the past to reproduce the AD physiopathology by injecting the beta-amyloid peptide directly inside the animal brains. These attempts have never been conclusive for different reasons, such as the type of peptide used, the localization of the injection, the concentration and the timing of the injection and also, in all these studies, the beta-amyloid peptide was injected alone. Previous animal models do not represent an appropriate animal model for the efficient development of preventive, therapeutic or diagnostic means for neurologic diseases. Therefore, there is a need for novel animal models.

### FIELD OF THE INVENTION

This invention relates to animal models simulating neurological disease.

### SUMMARY OF INVENTION

The present invention relates to a non-human animal model simulating neurologic disease, as well as methods and compositions for preparation of the animal model, and methods for using it. The non-human animals simulating neurologic disease are obtained by perfusing the animals with Aβ in combination with at least one pro-oxidative compound and at least one anti-oxidant inhibitor. In addition, the animals may also be perfused with a phosphatase inhibitor and/or a pro-inflammatory compound. The resulting non-human mammals develop neurologic disease in the brain. The animal models may be used in screening protocols for agents, which can be used for the development of preventive, therapeutic, and/or diagnostic means for neurologic diseases.

In one embodiment, a pharmacological animal model for AD may be developed by inducing both histopathological modifications of brain tissue and memory impairment, which creates a microenvironment in the brain similar to that hypothesized to occur in the AD brain.

### BRIEF DESCRIPTION OF DRAWINGS

FIG. 1A is a bar graph of a Morris watermaze score obtained by the various groups of rats. Results are shown as means±SD (n=8). ***p<0.001 compared to control group.

FIG. 1B is a bar graph phosphorylated Tau levels in rat CSF, means±SD (n=6-8). ***p<0.001 compared to control group.

FIG. 2A is a photomicrograph of a Campbell-Switzer staining of amyloid plaques and neurofibrillary tangles in the hippocampus of the control group (×40). Activated microglia phagocyting Aβ₄₂ (yellow arrow), neuritic plaques (red arrow), neurons containing NFT (blue arrow), vascular amyloidosis (black arrow) (applicable to all photomicrographs in FIG. 2).

FIG. 2B is a photomicrograph of a Campbell-Switzer staining of amyloid plaques and neurofibrillary tangles in the hippocampus of the FAB group (×40).

FIGS. 2C-2E are photomicrographs of a Campbell-Switzer staining of amyloid plaques and neurofibrillary tangles in the CA1 of the FAB group (×400).

FIG. 2F is a photomicrograph of a Campbell-Switzer staining of amyloid plaques and neurofibrillary tangles in the CA2 of the FAB group (×400).

FIG. 2G is a photomicrograph of a Campbell-Switzer staining of amyloid plaques and neurofibrillary tangles in the CA3 of the FAB group (×400).

FIGS. 2H-2I are photomicrographs of a Campbell-Switzer staining of amyloid plaques and neurofibrillary tangles in the dentate gyrus of the FAB group (×400).

FIG. 2J is a photomicrograph of a Campbell-Switzer staining of amyloid plaques and neurofibrillary tangles in the cingulate cortex of the control group (×40).

FIG. 2K is a photomicrograph of a Campbell-Switzer staining of amyloid plaques and neurofibrillary tangles in the cingulate cortex of the FAB group (×40).

FIG. 2L is a photomicrograph of a Campbell-Switzer staining of amyloid plaques and neurofibrillary tangles in the temporal cortex of the control group (×400).

FIGS. 2M-2O are photomicrographs of a Campbell-Switzer staining of amyloid plaques and neurofibrillary tangles in the temporal cortex of the FAB group (×400).

FIG. 2P is a photomicrograph of a Campbell-Switzer staining of amyloid plaques and neurofibrillary tangles in the parietal cortex of the FAB group (×1000).

FIGS. 3A-3D are photomicrographs of phosphorylated-Tau immunostaining in control (3A and 3C) and FAB (3B and 3D) rats. Immunoreactive phosphorylated Tau was found in CA1 (3B) and in dentate gyrus (3D) neurons (yellow arrows) compared to controls (3A and 3C).

FIGS. 3E-3H are photomicrographs of Aβ₄₂ immunostaining in control (3E and 3G) and FAB (3F and 3H) rats. FAB rats display a strong Aβ₄₂ immunoreactivity in CA1 (3F), in CA3 (3H) and in cingulate cortex (3F) compared to controls (3E and 3G).

FIGS. 4A-4C are photomicrographs of GFAP immunostaining of activated astrocytes in CA1 of control (4A) and FAB (4B and 4C) rats.

FIGS. 5A-5H are photomicrographs depicting neuronal death by De Olmos amino cupric silver and cresyl violet staining. Dead cells stain in black with the amino cupric silver method in the CA1, CA2, CA3, and dentate gyrus (5B) areas of FAB rats compared to control rats (5A). The loss of neurons was confirmed by the cresyl violet staining as shown by a decrease of staining density observed in CA1, CA3 and dentate gyrus crest of FAB rats (5D) compared to control (5C). An important neuronal loss was also observed in the temporal cortex of FAB rats as shown by the two different staining methods (5F and 5H) compared to control (5E and 5G).

### DETAILED DESCRIPTION

The present invention relates to a non-human animal model, preferably a monkey, dog or rodent, such as a mouse or rat, or other animal, which is naturally able to perform learning and memory tests, together with methods and compositions for preparing and using the animal. The animal is co-infused with Aβ and at least one pro-oxidative compound and at least one anti-oxidant inhibitor capable of triggering in the animal brain the physiopathological modifications observed in human neurologic disease. As used herein, Aβ refers to Aβ₄₂, peptide fragments of Aβ₄₂, such as Aβ₁₋₄₀ or Aβ₂₄₋₃₅, for example, or peptidomimetics that mimick amyloid. In addition, the animals may also be perfused with a phosphatase inhibitor and/or a pro-inflammatory compound. After perfusion, the animal develops a neurologic disease within a short period of time, generally within 30 days.

The present invention is further directed to a method of screening a compound useful in the development of preventive, therapeutic, and/or diagnostic means for neurologic diseases. Compounds of interest may be administered to the infused animal to evaluate the effectiveness of the compound in reversing a neurologic disorder.

The term “treat” or “treatment” or “therapy” or “therapeutic” as used herein refers to any treatment of a disorder or disease associated with a disease or disorder related to neurologic disease, including but not limited to AD, neurotoxicity, or beta-amyloid-induced neurotoxicity, in a subject, and includes, but is not limited to, preventing the disorder or disease from occurring in a subject who may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, halting the symptoms of the disease or disorder. As used herein, “neurologic disease” is intended to encompass all disorders and/or diseases stated above.

The term “prevent” or “prevention,” in relation to a disease or disorder related to neurologic disease, in a subject, means no disease or disorder development if none had occurred, or no further disorder or disease development if there had already been development of the disorder or disease.

Unless otherwise defined, all technical and scientific terms used herein have the same meaning as commonly understood by one of ordinary skills in the art to which this invention belongs.

To induce neurologic disease, non-human animals may be directly infused in the left ventricle for four weeks with a solution containing either Aβ alone or in combination with pro-oxidative compounds and anti-oxidant inhibitors. Aβ₄₂ is the peptide mostly involved in AD. As mentioned above, peptide fragments of Aβ₄₂, such as A₁₋₄₀ or Aβ₂₄₋₃₅, for example, or peptidomimetics that mimick amyloid may also be used in the present invention. Pro-oxidative compounds utilized in combination with Aβ for triggering the physiopathological modifications observed in human AD may include ferrous sulfate (FeSO₄), copper sulfate, cobalt sulfate, manganese sulfate, and zinc sulfate, for example. Ferrous sulfate is pro-oxidative and a constituent of the neuritic plaque. Further, inhibitors of anti-oxidant defenses of the brain that assist in triggering the physiopathological modifications observed in human AD may include buthionine sulfoximine (BSO), for example. Buthionine sulfoximine is an inhibitor of glutathion synthesis which may be used to lower the anti-oxidative defense of the brain. In addition, any chemical or pharmacological agent capable of lowering the anti-oxidative defenses of the brain may mimic the effect of BSO and, therefore, be of interest in an animal model. One of the purposes of the use of FeSO₄ and BSO is to create conditions favorable to Aβ toxicity. Further, in addition to the administration of Aβ, a pro-oxidative compound, and an anti-oxidant inhibitor phophatase inhibitors and pro-inflammatory compounds may also be administered. The phosphatase inhibitor may include okadaic acid, 1-nor-okadaone, bioallethrin, calycullin A, cantharidic acid, cantharidin, cypermethrin, delatamethrin, endothall, endothall thioanhydride, fenvalerate, okadol, permethrin, phenylarsine oxide, pyrophosphate, sodium floride, and vanadate, for example. Okadaic acid inhibits the dephosphorylation of the Tau protein, leading to a hyperphosphorylated state of this protein and, in turn, to the formation of NFT, the second histological hallmark of AD. The pro-inflammatory compound may include TNF-α, IL-6, and IL-1b, for example. TNF-α reproduces the inflammatory process documented in AD. These compounds described above may be co-administered separately or together and simultaneously or sequentially. Moreover, aluminum may be used to induce Alzheimer-like physiopathology. The compounds are used in a “pharmacologically effective amount.” This means that the concentration of the compounds administered are such that in the administered combination it results in the AD-inducing level of compounds delivered over the term that the compounds are administered.

After the animals are infused with a combination of the compounds mentioned above, the animals may be evaluated to determine the presence of neurologic disease by using a species appropriate neurobehavioral test. For example, studies of locomotor/exploratory behavior in mice is a standard means of assessing the neuropsychology (File and Wardill, (1975) Psychopharmacologia (Berl) 44:53-59; Loggi et al., (1991) Pharmacol. Biochem. Behav. 38:817-822). For example, for mice, the “corner index” (CI) test is used. This is a quick and simple neurobehavioral test to screen animals for evidence of brain pathology. The neuropathology of the animals also is evaluated. To perform the CI test, a test mouse, held by the tail, is placed in the center of a clean cage that is otherwise identical to its home cage. The number of times the mouse sniffs the corners of the test cage during the first 30 seconds after it was placed into that cage is recorded as the CI. Animals which are obviously moribund before attaining the CI criteria and animals which develop witnessed seizures also are diagnosed as ill. To control the variations in diurnal activity, all animals may be tested between the times of 14:30 and 18:30.

For rats, the Morris watermaze test (Morris, (1984) J. Neurosci. Meth. 11:47), may be used. A modified version of this test can be used with mice. In addition to the Morris watermaze test, the Y-maze test may be used to explore spatial memory deficit, the open field test to measure an initial neophobic response and subsequent exploratory behavior, and the spontaneous non-matching-to-sample test which assesses the spontaneous object recognition (Ennaceur and Delacour, 1988 Aggleton, 1993).

Brain regions known to be affected by the syndrome of interest are particularly reviewed for changes. When the disease of interest is AD, the regions reviewed include the cortico-limbic region, the amygdala, the olfactory bulbs, and the conditions monitored include gliosis, neuronal death, NFT formation, cholinergic neurotransmission impairment, neurosteroid concentration in blood and cerebrospinal fluid, alteration in gene expression in brain structures, changes in glucose uptake and utilization and Aβ plaque formation. However, in strains of animals which are not long-lived, not all behavioral and/or pathological changes associated with a particular disease may be observed. It is unlikely that the short life of this animal species would mask the appearance of more delayed symptoms. The tests involved to answer this question are identical to those already used or scheduled to characterize the model itself.

The animals of the present invention may be used to screen compounds of interest, e.g. antioxidants such as vitamin E or lazaroids, thought to prevent and/or treat AD. An animal may be administered the compound of interest, and a reduced incidence or delayed onset of neurologic disease, as compared to untreated animals, is detected as an indication of protection. The indices used preferably are those which can be detected in a live animal, such as changes in performance on learning and memory tests. The effectiveness can be confirmed by effects on pathological changes when the animal dies or is sacrificed. The animals further can be used to screen compounds of interest thought to improve or cure AD. An animal with neurologic disease is treated with the material of interest, and a delayed death, or improvement in neurobehavior, gliosis, or glucose uptake/utilization, as compared to untreated animals with neurologic disease, is detected as an indication of amelioration or cure.

The animals of the present invention may also be used to screen a compound or test a situation, e.g. oxidants or head trauma, suspected of accelerating or provoking AD, by exposing the animal to the compound or situation and determining neurobehavioral decline, premature death, gliosis, and diminished glucose uptake/utilization as indicators of the capacity of the test material or situation to induce AD. The method further can include screening therapeutic agents by exposing animals to a compound or situation suspected of provoking AD and evaluating the effect of the therapeutic agent.

The animals of the present invention may further be used to characterize and highlight parameters which may be of interest to develop a diagnostic applicable to diagnose a neurological disease in a subject. The animals may be used as to detect changes in fluids, tissues, etc. that may be used to develop a diagnostic assay in humans. This may be particularly valuable in the case of early detection before the appearance of symptoms indicating cognitive impairment.

It is believed that one skilled in the art, based on the description herein, can utilize the present invention to its fullest extent. All publications recited herein are hereby incorporated by reference in their entirety. The following specific examples are therefore to be construed as merely illustrative, and not limitative of the remainder of the disclosure in any way whatsoever.

### EXAMPLE I

Inducing a Neurologic Disease in a Non-Human Animal. Animals: Long-Evans male rats weighing 300-325 grams were housed following a natural day-night cycle with food and water ad libitum.

Morris watermaze protocol: Before the surgery, the rats were trained on a standard Morris spatial navigation task in a black water tank (200 cm diameter). The water was rendered opaque by water-miscible non-toxic white paint (Crayola Inc.). The rats were placed in four different, randomly assigned, start positions and trained to find an invisible platform (20 cm diameter) at a fixed position in the middle of the water tank. The water temperature was about 24° C. A trial lasted until a rat found the platform or until 120 seconds elapsed. If a rat did not find the platform within 120 seconds, it was placed on the platform for 20 seconds and then removed from the water tank. Rats were trained for four consecutive days, four trials a day, with 30 minutes between subsequent trials. Surgery procedures were performed on the fifth day. Rats were tested again for memory retention at the end of the four weeks-perfusion period.

Surgical procedure: Anesthetized rats (equitesin, 3 ml/kg, i.p.) were placed on a stereotaxic frame. Using the electrode micromanipulator, the outlet of an osmotic micropump (Durect Corp., Cupertino, Calif.) was implanted into the left ventricle following the coordinates D=3.4 mm, L=1.4 mm and AP=0.92 mm prior to the bregma. The tank of the osmotic pump was implanted in a subcutaneous pocket in the midscapular area of the back of the rat. After surgery rats were put on a heating blanket for recovery. During the whole procedure, the body temperature was monitored and kept stable at 37° C.

The brains were removed four weeks later, after rats have been intracardiacally perfused, first, with a washing solution (NaCl 8 g/l, Dextrose 4 g/l, Sucrose 8 g/l, Calcium chloride 0.23 g/l, Sodium cacodylate anhydrous 0.25 g/l, in deionized water) and, second, with fixative cacodylate buffer (Sucrose 40 g/l, Paraformaldehyde 40 g/l, Sodium cacodylate anhydrous 10.72 g/l in deionized water). Brains were stored in the fixative cacodylate buffer until being processed.

Perfusion: Rats were divided into six groups regarding the composition of the solution contained in the osmotic micropump tank, as provided in Table 1. Group I served as the control group and received only artificial cerebrospinal fluid (“CSF”).

TABLE 1

| | | | Buthionine | | |
|---|---|---|---|---|---|
| | Aβ₄₂ | Ferrous | sulfoximine | Okadaic | TNFα |
| | (mM) | sulfate (mM) | (mM) | acid (μM) | (pM) |
| Group I | | | | | |
| Group II | 15 | | | | |
| Group III | 15 | 1 | | | |
| Group IV | 15 | 1 | 12 | | |
| Group V | 15 | 1 | 12 | 10 | |
| Group VI | 15 | 1 | 12 | 10 | 43.75 |

Immunohistochemistry: In order to be processed for immunohistochemistry and staining, sixteen brains were embedded at once in a gelatin block and cut in 40 μm thick slices (Neurosciences Associates, Knoxyille, Tenn.). Forty μm thick brain slices were processed with different primary antibodies: anti phosphorylated Tau protein recognizing the phosphorylated Ser202 (BioSource International, Inc., Camarillo, Calif.), anti-GFAP (Santa Cruz Biotechnology, Inc., Santa Cruz, Calif.) for activated astrocytes, anti-Aβ₄₂ specifically recognizing the C-terminus 33-42aa of the peptide (Signet Laboratories, Inc., Dedham, Mass.) and anti-CD11b (clone OX42) (Novus Biologicals, Inc.) for activated microglia.

ELISA: Hyperphosphorylated Tau protein was measured in the CSF using the Elisa kit INNOTEST™ (Innogenetics, Belgium).

Stainings: Consecutive 40 μm thick brain slices were stained with (1) cresyl violet 0.5% pH=3.7 for 10 minutes before being mounted with aqueous mounting medium; (2) the De Olmos amino cupric silver stain; and (3) Campbell-Switzer silver staining. Cresyl violet stains Nissl bodies and nucleus in neurons. Nissl bodies are lost when the neuron is dead, leading to a decrease or a lack of staining. Cell bodies, dendrites, axons and synaptic terminals of dead neurons appear stained in black with the De Olmos amino cupric silver stain. The Campbell-Switzer silver staining reveals specifically the neuritic plaques and the NFT (Neuroscience Associates, Knoxyille, Tenn.) (Switzer III et al., U.S. Pat. No. 5,192,688 (1993)).

Statistical analysis: Statistical analysis of the Morris watermaze data was performed using a one-way ANOVA followed by a multiple comparisons Dunnett test.

Results: Administration of Aβ₄₂ alone was not sufficient to induce memory impairment and the appearance of phosphorylated Tau protein in the CSF, a well-characterized marker of AD in humans. (Green A. J. E. Cerebrospinal fluid brain-derived proteins in the diagnosis of Alzheimer's disease and Creutzfeld-Jakob disease. Neuropathol. Appl. Neurobiol. 28, 427-440 (2002); Mulder C., et al. CSF markers related to pathogenetic mechanisms in Alzheimer's disease. J. Neural. Transm. 109, 1491-1498 (2002)). These data also question Aβ₄₂, and the amyloidogenic hypothesis, as the sole culprit of AD.

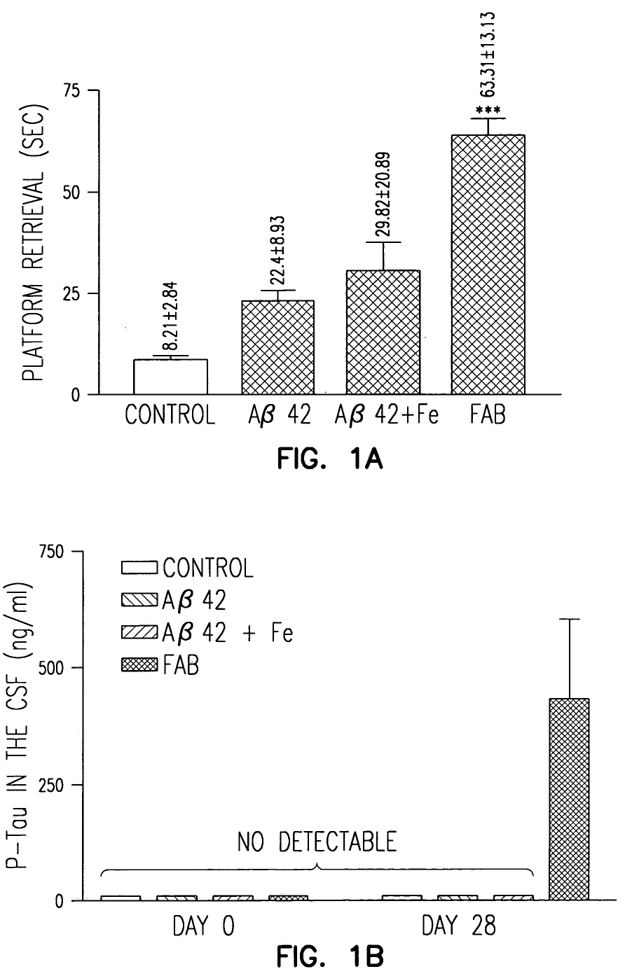

Ferrous was added to the infusion solution because iron is a constituent of the neuritic plaques (Lovell M. A., et al., Iron and zinc in Alzheimer's disease senile plaques. J. Neurol. Sci. 158(1), 47-52 (1998)) and its presence is responsible for changing steroid formation in the brain, thus altering the endocrine balance of the tissue. (Brown R. C., et al. Oxidative stress-mediated DHEA formation in Alzheimer's disease pathology. Neurobiol. Aging 24(1), 57-65 (2003)). However, Aβ₄₂ with ferrous did not significantly affect the memory of the animals and did not induce the presence of phosphorylated Tau protein in CSF (FIG. 1B).

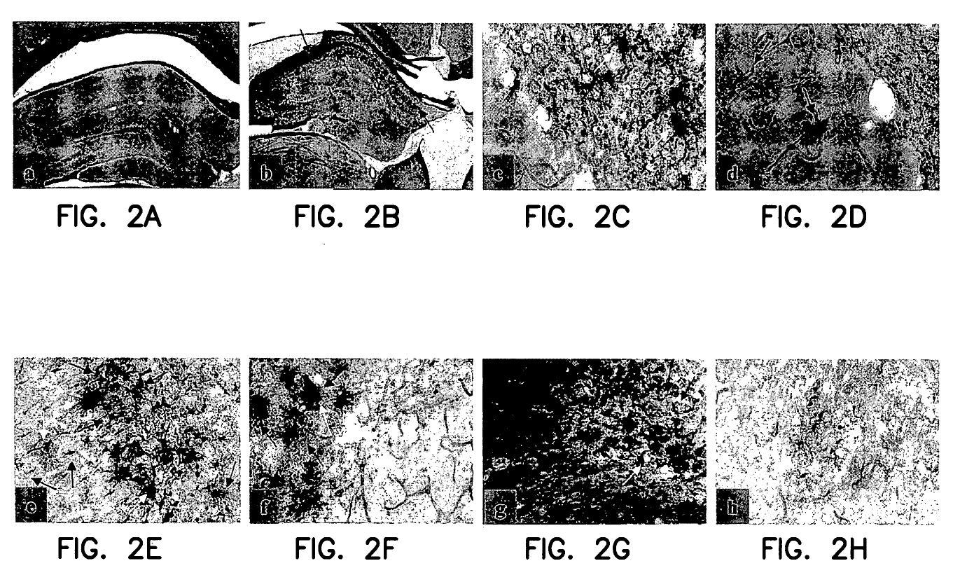

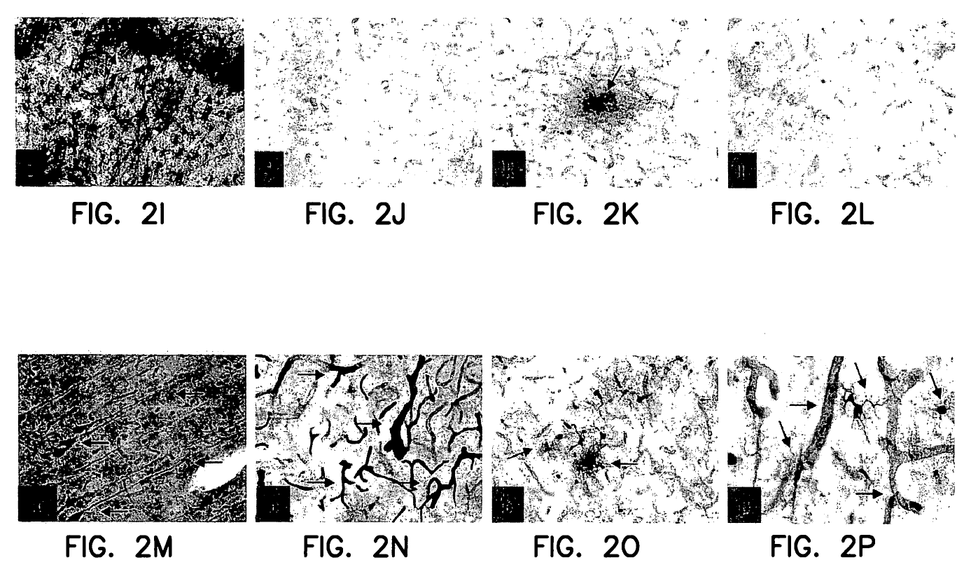

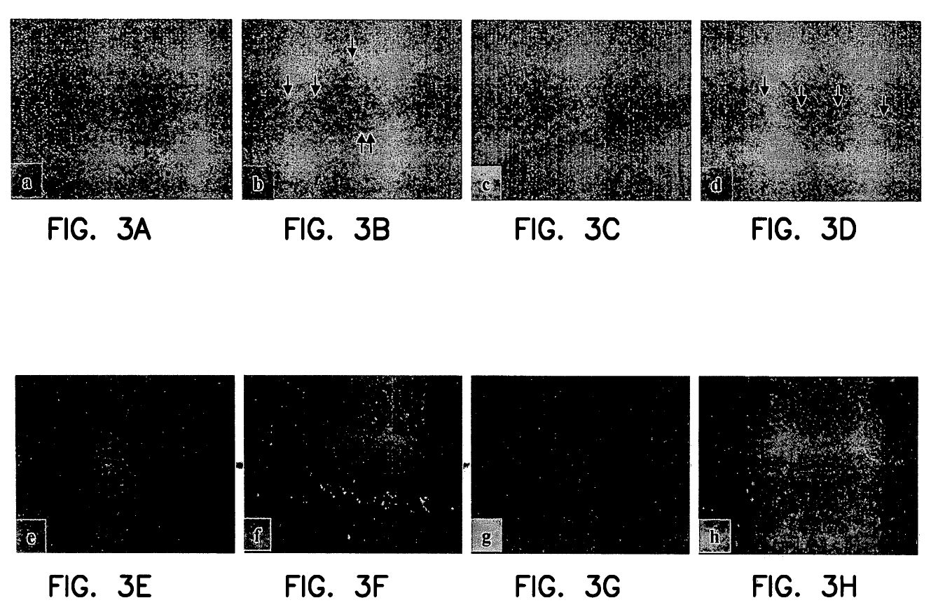

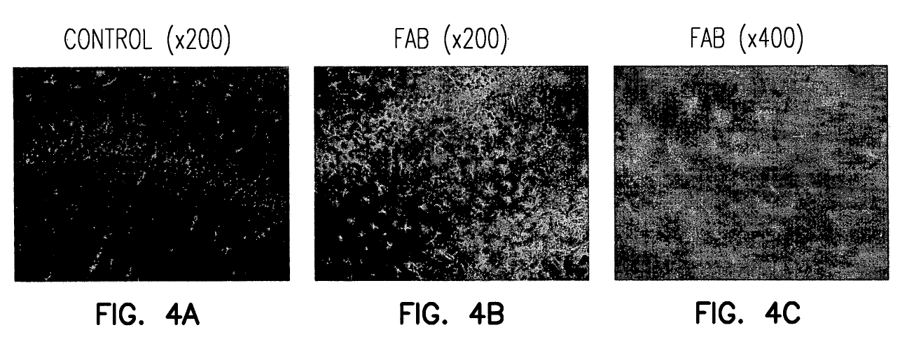

This lack of effect of the Aβ₄₂/ferrous solution was corrected by lowering the anti-oxidative defense of the brain and in turn inducing an oxidative stress. This was achieved by the addition of buthionine sulfoximine (BSO), an inhibitor of the glutathion synthesis, in the Aβ₄₂/ferrous solution. The Fe²⁺-Aβ₄₂-BSO (FAB) infused rats displayed a significant increase of the latency to retrieve the platform in the Morris watermaze test showing an alteration of memory processes (FIG. 1A). The FAB-induced memory impairment was accompanied by a significant increase of the concentration of the hyperphophorylated Tau protein in the CSF (FIG. 1B), reproducing exactly what has been described in AD patients. (Green and Mulder et al., above). As in human, this parameter may be used in the diagnosis and the assessment of drug-candidate activity. In addition, the memory impairment was associated with AD-like histological modifications as brought to the fore by various histological methods. After a one-month infusion, the FAB rats developed neuritic plaques mostly in the CA1 area of the hippocampus (FIGS. 2C-2E), a region well known to be highly sensitive to Aβ₄₂ toxicity, as revealed by the Campbell-Switzer silver method. Plaques were also observed in the CA2 and CA3 areas of the hippocampus (FIGS. 2F and 2G, respectively), and also in the cingulate and temporal cortex (FIGS. 2K and 2M-2O, respectively). The presence of Aβ₄₂ in these brain areas was further confirmed using an antibody raised specifically against the C-terminus (amino acids 33-42) of Aβ₄₂ (FIGS. 3E-3H). In addition, the specific Campbell-Spitzer staining evidenced phagocytic microglial cells containing Aβ₄₂ and intra-neuronal amyloidosis in the hippocampus.

In agreement with the increase of hyperphosphorylated Tau protein levels in CSF, FAB rats displayed intense NFT staining in the different structures of the hippocampus, as demonstrated by the Campbell-Switzer stain (FIGS. 2A-2P) and confirmed by immunostaining (FIGS. 3A-3D). NFT containing neurons were also observed in the parietal and cingulate cortex (FIGS. 2P, 2J, and 2K, respectively) (Sigurdsson E. M., et al., Bilateral injections of amyloid-β₂₅₋₃₅ into amygdala of young Fischer rats: behavioral, neurochemical, and time dependent histopathological effects. Neurobiol. Aging 18(6), 591-608 (1997)), which demonstrates the ability of Aβ₄₂ to induce NFT formation.

The finding that Aβ₄₂ induces NFT formation in young adult rat brain only when it is associated to an important oxidative stress reproduces, at least in part, the intracranial conditions in aged brain. (Floyd R. A. & Hensley K. Oxidative stress in brain aging: implications for therapeutics of neurodegenerative diseases. Neurobiol. Aging 23, 795-807 (2002)). The physiopathology of AD has a vascular component similar to what has been described for vascular dementia. (De La Torre J. C. Vascular basis of Alzheimer's pathogenesis. Ann. N.Y. Acad. Sci. 977, 196-215 (2002); Miyakawa T. Vascular pathology in Alzheimer's disease. Ann. N.Y. Acad. Sci. 977, 303-305 (2002)). This vascular component may be used in the prognosis and diagnosis of the disease. Vascular amyloid deposits have been described in the vasculature of post mortem AD human brain specimens. However, transgenic mice expressing a high level of neuritic plaques do not develop vascular amyloidosis. (Janus C. (2000); Janus C. & Westaway D. (2001)). In contrast to transgenics, the FAB model displays vascular amyloidosis in the temporal cortex as shown by the Campbell-Switzer stain (FIGS. 2M-2O), further strengthening the validity of this model.

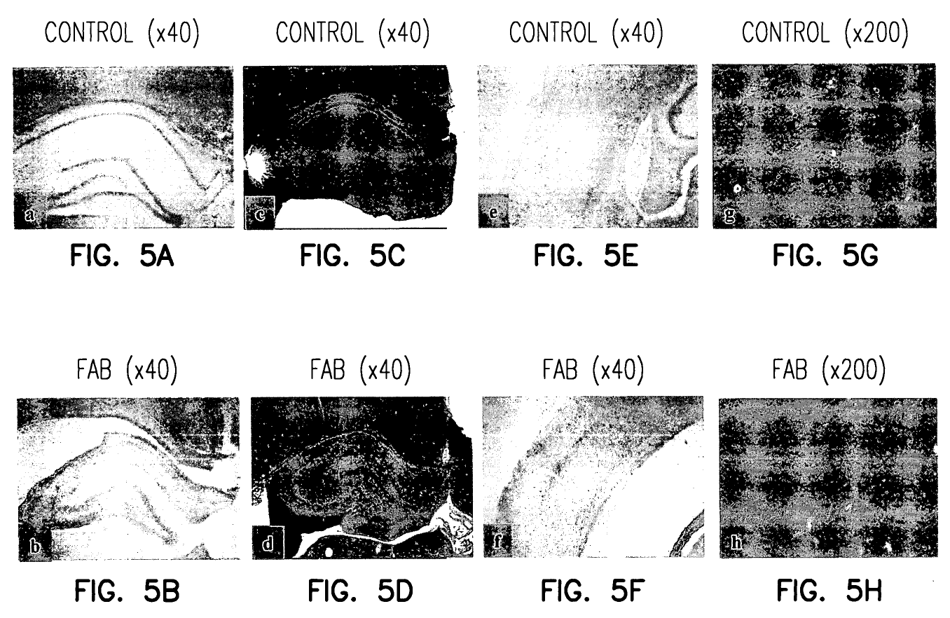

The massive astrogliosis and microgliosis found in the CA1, CA3 and dentate gyrus areas of the hippocampus (FIGS. 2C-E, 2G, 2H-2I, and 4B-4C), mostly in the vicinity of the neuritic plaques, reflect an important inflammatory process occurring in FAB-infused rat brains. These histopathological modifications described in FAB-infused rat brains were accompanied by important neuronal death detected by both amino cupric silver and cresyl violet staining (FIGS. 5A-5H). Neuronal death occurred essentially in the CA1 hippocampal area although dead neuronal bodies were also observed in the CA3 area and in the cingulate cortex.

The data presented indicate that continuous four week infusion into the left ventricle of a young adult rat of a solution containing the amyloidogenic peptide Aβ₄₂, the pro-oxidative cation Fe²⁺ and the glutathione synthesis inhibitor BSO has the ability to create the memory loss, increased phosphorylated Tau protein levels in CSF, and the histopathological profile, including neuronal loss, seen in AD. Thus, the FAB rat model provides an animal model reflecting AD pathology, useful in understanding the molecular mechanisms involved in the onset and progression of the disease, and a valuable tool for the fast screening of novel means for diagnosis, prevention and therapy of AD.

Any modification and additions in the FAB solution that are based on the above described concept are also encompassed within this invention.

### EXAMPLE 2

Testing for Drugs That Prevent Neurologic Disease. The animals of the present invention may be used to test compounds for the ability to confer protection against the development of neurologic disease, such as AD. An animal exhibiting a neurologic disease is treated with a test compound in parallel with an untreated control animal exhibiting the neurologic disease. A comparatively lower incidence of the neurologic disease in the treated animal is detected as an indication of protection. Treated and untreated animals are analyzed for diminished learning/exploratory/locomotor behavior (CI test), as well as diminished 2-deoxyglucose uptake/utilization, neuronal death, cholinergic neurotransmission impairment, neuritic plaques, NFT formation and hypertrophic gliosis in the cortico-limbic structures of the brain. To determine if a treatment can prevent or delay the onset of disease, half of the animals, for example mice, in a litter from a line of mice known to develop neurologic illness may be randomly assigned to receive the treatment, and the other half to receive a placebo, beginning at an age prior to the earliest known onset of disease for the given line of mice. The number of litters to be used will depend upon the magnitude of the differences observed between treated and untreated mice.

Mice are observed daily; their diagnosis is facilitated by the use of the CI test, which is administered three times per week by individuals blinded to the experimental groups. Survival curves and mean ages of disease onset and death are calculated from the accumulated clinical data.

Pre-clinical results are corroborated by performing neuropathologic and glucose-uptake studies in samples in the experimental and control groups. Gliosis is evaluated in immunohistologic studies using antibodies raised against the glial fibrillary acidic protein and the CD11b (clone OX-42). Glucose-uptake studies are performed using a modification of the Sokoloff method described by Chmielowska, et al., (1986) Exp. Brain Res. 63:607.

### EXAMPLE 3

Testing for Drugs That Treat Neurologic Disease. The animals of the present invention may be used to test compounds for the ability to improve or cure neurologic disease. An animal exhibiting the neurologic disease is treated with a test material in parallel with an untreated control animal exhibiting the neurologic disease. A comparatively delayed death, or an improvement in the neurobehavioral, pathologic, or functional indications of the disease is detected as an indication of protection. Treated and untreated animals are analyzed for diminished learning/exploratory/locomotor behavior, as well as diminished 2-deoxyglucose uptake/utilization, neuronal death, cholinergic neurotransmission impairment, neuritic plaques, NFT formation and hypertrophic gliosis in the cortico-limbic structures of the brain.

As demonstrated by the above results, the pre-clinical and pathologic findings in non-human FAB mammals show an unexpected, but striking parallel to those in humans with neurologic disorders such as AD. The correlative appearance of behavioral, biochemical and pathological abnormalities reminiscent of AD affords new opportunities for exploring the pathophysiology and neurobiology of AD in non-human animals.

A number of embodiments of the invention have been described. Nevertheless, it will be understood that various modifications may be made without departing from the spirit and scope of the invention. Accordingly, other embodiments are within the scope of the following claims, and as various changes can be made to the above compositions, formulations, combinations, and methods without departing from the scope of the invention, it is intended that all matter contained in the above description be interpreted as illustrative and not in a limiting sense. All patent documents and references listed herein are incorporated by reference.

1. A non-human animal having a neurologic disease induced by the process of:
perfusing the non-human animal with a pharmacologically effective amount of a combination of an Aβ compound, at least one pro-oxidative compound, and at least one anti-oxidant inhibitor, wherein the perfusion produces impaired performance of the animal in memory and learning tests and induces abnormal neuropathology in a brain of the animal, wherein said impaired performance and abnormal neuropathology are in comparison with control non-human animals.

2. The non-human animal of claim 1, wherein the Aβ compound comprises Aβ₄₂.

3. The non-human animal of claim 1, wherein the Aβ compound comprises a peptide fragment of Aβ₄₂.

4. The non-human animal of claim 3, wherein the peptide fragment of Aβ₄₂ comprises at least one of Aβ₁₋₄₀ or Aβ₂₄₋₃₅.

5. The non-human animal of claim 1, wherein the Aβ compound comprises a peptidomimetic that mimicks Aβ₄₂.

6. The non-human animal of claim 1, wherein the at least one pro-oxidative compound is selected from the group consisting of ferrous sulfate, copper sulfate, cobalt sulfate, manganese sulfate, and zinc sulfate.

7. The non-human animal of claim 1, wherein the at least one pro-oxidative compound comprises ferrous sulfate.

8. The non-human animal of claim 1, wherein the at least one anti-oxidant inhibitor comprises buthionine sulfoximine.

9. The non-human animal of claim 1, wherein the process further comprises perfusing the non-human animal with an effective amount of a phosphatase inhibitor.

10. The non-human animal of claim 9, wherein the phosphatase inhibitor is selected from the group consisting of okadaic acid, 1-nor-okadaone, bioallethrin, calycullin A, cantharidic acid, cantharidin, cypermethrin, deltamethrin, endothall, endothall thioanhydride, fenvalerate, okadol, permethrin, phenylarsine oxide, pyrophosphate, sodium fluoride, and vanadate.

11. The non-human animal of claim 9, wherein the phophatase inhibitor comprises okadaic acid.

12. The non-human animal of claim 1, wherein the process further comprises perfusing the non-human animal with an effective amount of a pro-inflammatory compound.

13. The non-human animal of claim 12, wherein the pro-inflammatory compound is selected from the group consisting of TNF-α, IL-6, and IL-1b.

14. The non-human animal of claim 12, wherein the pro-inflammatory compound comprises TNF-α.

15. A method for inducing a neurologic disease in a non-human animal, comprising:
perfusing the non-human animal with a pharmacologically effective amount of a combination of an Aβ compound, at least one pro-oxidative compound, and at least one anti-oxidant inhibitor.

16. The method of claim 15, wherein the Aβ compound comprises Aβ₄₂.

17. The method of claim 15, wherein the Aβ compound comprises a peptide fragment of Aβ₄₂.

18. The method of claim 17, wherein the peptide fragment of Aβ₄₂ comprises at least one of Aβ₁₋₄₀ or Aβ₂₄₋₃₅.

19. The method of claim 15, wherein the Aβ compound comprises a peptidomimetic that mimicks Aβ₄₂.

20. The method of claim 15, wherein the at least one pro-oxidative compound is selected from the group consisting of ferrous sulfate, copper sulfate, cobalt sulfate, manganese sulfate, and zinc sulfate.

21. The method of claim 15, wherein the at least one pro-oxidative compound comprises ferrous sulfate.

22. The method of claim 15, wherein the at least one anti-oxidant inhibitor comprises buthionine sulfoximine.

23. The method of claim 15, further comprising perfusing the non-human animal with an effective amount of a phosphatase inhibitor.

24. The method of claim 23, wherein the phosphatase inhibitor is selected from the group consisting of okadaic acid, 1-nor-okadaone, bioallethrin, calycullin A, cantharidic acid, cantharidin, cypermethrin, deltamethrin, endothall, endothall thioanhydride, fenvalerte, okadol, permethrin, phenylarsine oxide, pyrophosphate, sodium fluoride, and vanadate.

25. The method of claim 23, wherein the phophatase inhibitor comprises okadaic acid.

26. The method of claim 15, further comprising perfusing the non-human animal with an effective amount of a pro-inflammatory compound.

27. The method of claim 27, wherein the pro-inflammatory compound is selected from the group consisting of TNF-α, IL-6, and IL-1b.

28. The method of claim 27, wherein the pro-inflammatory compound comprises TNF-α.

29. A method of screening for an agent that ameliorates symptoms of a neurologic disease, said method comprising:
comparing performance on memory and learning tests of a first non-human animal contacted with the agent with that of a second non-human animal not contacted with the agent, wherein the first and said second non-human animals have been co-infused with a pharmacologically effective amount of Aβ, at least one pro-oxidative compound, and at least one anti-oxidant inhibitor wherein the co-infusion produces impaired performance on the memory and learning tests and abnormal neuropathology in a brain of the first and second non-human animals, wherein the impaired performance and the abnormal neuropathology are in comparison with control non-human animals, whereby an agent which ameliorates the symptoms is identified by superior performance of said first non-human animal in comparison with the second non-human animal on the memory and learning tests.

30. A method for screening for an agent useful for treating a neurologic disease, said method comprising:
comparing performance on memory and learning tests of a first non-human animal contacted with the agent with that of a second non-human animal not contacted with the agent, wherein the first and said second non-human animals have been co-infused with a pharmacologically effective amount of Aβ and at least one pro-oxidative compound, and at least one anti-oxidant inhibitor, wherein the co-infusion produces impaired performance on the memory and learning tests and abnormal neuropathology in a brain of the first and second non-human animals, wherein the impaired performance and the abnormal neuropathology are compared with control non-human animals; and comparing neuropathology in the brain of the first and the second non-human animal when said first non-human animal exhibits superior performance on the memory and learning tests compared with the second non-human animal, whereby an agent which is useful for treating a neurologic disease is identified by a decrease in neuropathologic findings in the first non-human animal in comparison with the second non-human animal.
